(19) United States
(12) Patent Application Publication
(10) Pub. No.: US 2017/0280985 A1
(43) Pub. Date: Oct. 5, 2017

(54) AUDITORY CANAL CLEANING TOOL AND AUDITORY CANAL OBSERVING TOOL

(71) Applicant: Coden Co., Ltd., Tokyo (JP)

(72) Inventors: YOSHIHARU KODA, Tokyo (JP); Kojiro KODA, Tokyo (JP)

(21) Appl. No.: 15/271,352

(22) Filed: Sep. 21, 2016

(30) Foreign Application Priority Data
Mar. 31, 2016 (JP) 2016-070964

(51) Int. Cl. A61B 1/227; A61B 1/015; A61F 11/00; A61B 1/07; A61B 1/12; A61B 1/00; A61B 1/05

(52) U.S. Cl. CPC A61B 1/227; A61B 1/00094; A61B 1/015; A61B 1/05; A61B 1/07; A61B 1/00142; A61B 1/127; A61B 1/128; A61F 11/006

(57) ABSTRACT

Foreign matters of variable size in the interior of one's auditory canal are removed infallibly.

An image capturing tube 220 adapted to be inserted into one's auditory canal to output an image of an interior of the auditory canal, a suction tube 230 provided along the image capturing tube 220 to suck a foreign matter in the interior of the auditory canal, and a main body unit 200 provided with the image capturing tube 220 and the suction tube 230 are included. The image capturing tube 220 includes a camera 240 adapted to capture the image of the interior of the auditory canal, and to a tip end of the suction tube 230, a dust collecting tube 232 is attached.

### BACKGROUND

### 1. Technical Field

The present invention relates to an auditory canal cleaning tool for removing foreign matters in the interior of one's auditory canal and an auditory canal observing tool for observing the interior of the auditory canal.

### 2. Description of Related Arts

Cleaning of the interior of one's auditory canal is required for each of small and medium animals as well as each of human beings. In the interior of the auditory canal, cerumen in various states is generated such as dry cerumen, wet cerumen, and dandruff-like cerumen. Especially in each of the small and medium animals, microscopic animals such as ticks and lice tend to parasitize the interior of the auditory canal. For this reason, to find abnormalities in the interior of the auditory canal of each of the human beings and small and medium animals in early stage, it is preferable to observe and clean the interior of the auditory canal periodically.

In a case in which a human being cleans the interior of his/her auditory canal, a general procedure is to insert an ear pick into the auditory canal and remove cerumen in strict accordance with intuition. However, moving the ear pick in the interior of the auditory canal on the principle of trial and error may inflict injury to the auditory canal with a tip end of the ear pick. Also, since foreign matters, such as cerumen, of variable size, exist, it is difficult to remove the foreign matters completely only with the ear pick.

An endoscopic auditory canal cleaning apparatus illuminating the interior of one's auditory canal and having an ear cavity thereof viewed to prevent injury to the auditory canal to facilitate removal of cerumen is disclosed in Japanese Patent Application Laid-Open No. 2004-358098.

### SUMMARY

However, in the endoscopic auditory canal cleaning apparatus disclosed in Japanese Patent Application Laid-Open No. 2004-358098, a user must hold a rod-like ear pick device with one hand and a display device with the other hand and remove foreign matters such as cerumen while looking at the display device. Thus, the endoscopic auditory canal cleaning apparatus does not provide good usability and leaves room for improvement in terms of the usability.

Also, there is a demand for infallible removal of foreign matters such as fine cerumen remaining in the interior of the auditory canal. However, the endoscopic auditory canal cleaning apparatus disclosed in Japanese Patent Application Laid-Open No. 2004-358098 does not have a function of sucking the foreign matters in the interior of the auditory canal. Thus, it is difficult for the endoscopic auditory canal cleaning apparatus disclosed in Japanese Patent Application Laid-Open No. 2004-358098 to meet the demand for infallible removal of foreign matters.

Further, in animal hospitals for small animals, there is a demand for observation of the interior of the auditory canal on a clear image. However, it is difficult for the endoscopic auditory canal cleaning apparatus disclosed in Japanese Patent Application Laid-Open No. 2004-358098 to have the interior of the auditory canal of each of the small animals, which tends to be more humid than that of each of the human beings, observed without misting a lens of a camera.

The present invention is accomplished to meet such demands in the conventional art, and an object thereof is to provide an auditory canal cleaning tool enabling foreign matters of variable size in the interior of one's auditory canal to be removed infallibly and an auditory canal observing tool enabling the interior of the auditory canal to be observed while a user is looking at an image of the interior of the auditory canal clearly displayed on a display.

An auditory canal cleaning tool according to the present invention to achieve the above-described object includes: an image capturing tube adapted to be inserted into one's auditory canal to output an image of an interior of the auditory canal; a suction tube provided along the image capturing tube to suck a foreign matter in the interior of the auditory canal; and a main body unit provided with the image capturing tube and the suction tube, and the image capturing tube includes a camera adapted to capture the image of the interior of the auditory canal, and to a tip end of the suction tube, a dust collecting tube is attached.

An auditory canal observing tool according to the present invention to achieve the above-described object includes: an image capturing tube adapted to be inserted into one's auditory canal to output an image of an interior of the auditory canal; and a main body unit provided with the image capturing tube, and the image capturing tube includes a camera adapted to capture the image of the interior of the auditory canal and a heater heating the camera.

The auditory canal cleaning tool according to the present invention can remove foreign matters of variable size in the interior of one's auditory canal infallibly by suction.

Also, the auditory canal observing tool according to the present invention, in which an auditory canal imaging device and a display device are separated, enables the interior of the auditory canal to be observed on a bright image even in a case in which the interior of the auditory canal is humid.

### BRIEF DESCRIPTION OF THE DRAWINGS

FIG. 1 is a configuration diagram of an auditory canal cleaning tool according to Embodiment 1;

FIG. 2 is a side view of a main body unit in FIG. 1;

FIG. 3 is a plan view of the main body unit in FIG. 1;

FIG. 4 illustrates a configuration of an image capturing tube, a suction tube, and a dust collecting tube in FIG. 1 and an attaching procedure of the dust collecting tube;

FIG. 5A is a configuration diagram of the dust collecting tube;

FIG. 5B is a rear view of the dust collecting tube in FIG. 5A;

FIG. 5C is a sectional view of the dust collecting tube in FIG. 5A;

FIG. 6 is a configuration diagram of a cross-section of the image capturing tube;

FIG. 7 illustrates relationship between a suction pressure adjusting hole and the suction tube in the main body unit;

FIG. 8 is a specific configuration diagram of a periphery of the suction pressure adjusting hole in FIG. 7;

FIG. 9 is a control block diagram of a case in which a camera and an image processing section in FIG. 1 are connected in a wired manner;

FIG. 10 is a control block diagram of a case in which the camera and the image processing section in FIG. 1 are connected in a wireless manner;

FIG. 11 illustrates an example of an image obtained in the auditory canal cleaning tool in FIG. 1;

FIG. 12 illustrates a modification example of a heater;

FIG. 13 illustrates a modification example of the dust collecting tube;

FIG. 14 is a configuration diagram of an auditory canal cleaning tool according to Embodiment 2;

FIG. 15 is a configuration diagram of an auditory canal observing tool described as Embodiment 3;

FIG. 16 is a configuration diagram of an auditory canal observing tool in a state of attaching an ear pick portion and a handle portion thereto; and

FIGS. 17A, 17B and 17C illustrate examples of images obtained by the auditory canal observing tool in FIG. 16.

### DETAILED DESCRIPTION

Hereinbelow, an auditory canal cleaning tool and an auditory canal observing tool according to the present invention will be described with reference to the drawings. The auditory canal cleaning tool will be described as [Embodiment 1] and [Embodiment 2]. The auditory canal observing tool will be described as [Embodiment 3]. The auditory canal observing tool in a state of attaching an ear pick portion and a handle portion thereto will be described as [Embodiment 4].

### Embodiment 1

Embodiment 1 is an auditory canal cleaning tool enabling a camera to be prevented from being misted and enabling foreign matters in the interior of one's auditory canal to be sucked.

### [Configuration of Auditory Canal Cleaning Tool]

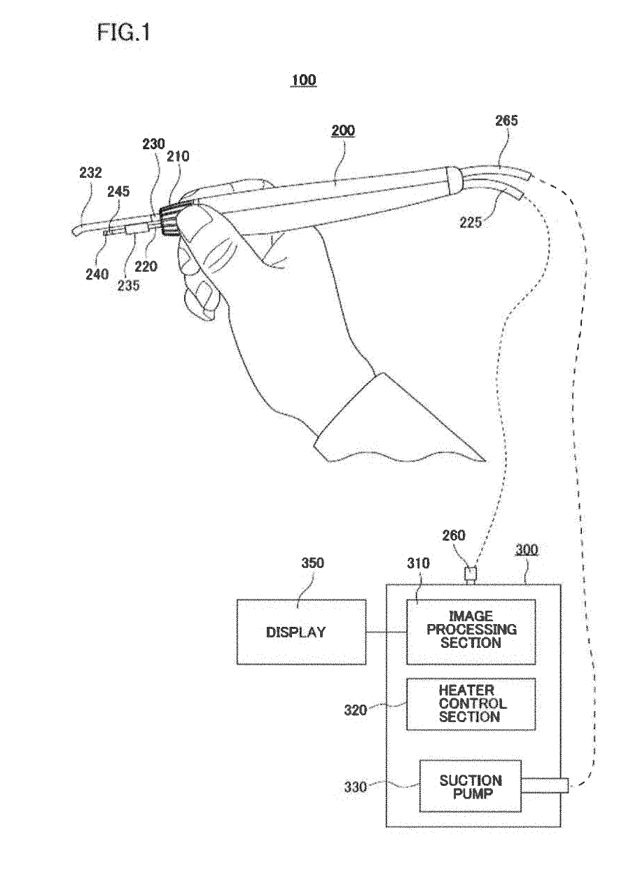

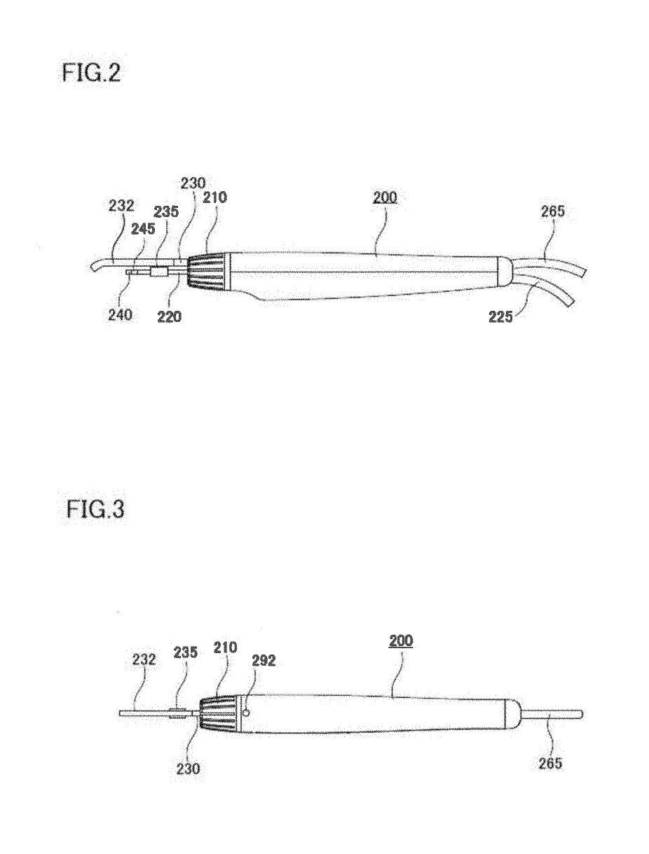

Hereinbelow, a configuration of an auditory canal cleaning tool according to Embodiment 1 will be described with reference to FIGS. 1 to 11 as needed.

As illustrated in FIG. 1, an auditory canal cleaning tool 100 includes a main body unit 200 and a control unit 300.

As illustrated in FIGS. 1 to 3, the main body unit 200 is formed in a rod-like long-elliptic shape and includes at a tip end on a front side thereof a tip end portion 210 that an operator holds with his/her fingers to support the main body unit 200. The tip end portion 210 has non-slip grooves to enable the operator to hold the main body unit 200 easily.

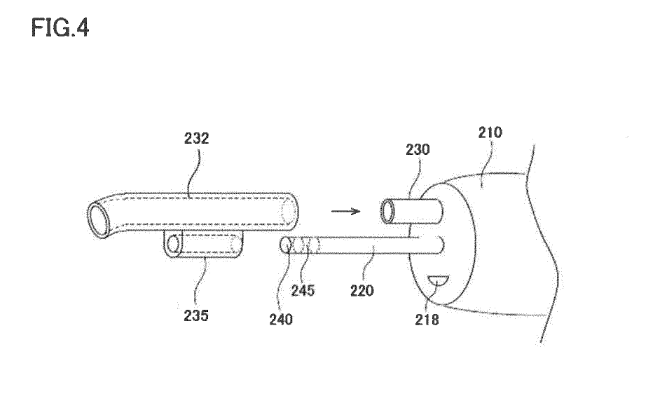

As illustrated in FIGS. 1 to 4, from the tip end portion 210 are projected an image capturing tube 220 adapted to be inserted into one's auditory canal to output an image of the interior of the auditory canal and a suction tube 230 provided along the image capturing tube 220 to suck foreign matters in the interior of the auditory canal (especially, refer to FIG. 4). In this manner, the main body unit 200 is provided with the image capturing tube 220 and the suction tube 230. The tip end portion 210 is provided with an ear pick portion attaching hole 218 adapted to attach a below-mentioned ear pick portion to the tip end portion 210 by inserting one end of the ear pick portion there into. Thus, the ear pick portion is detachable from the tip end portion 210.

To a tip end of the image capturing tube 220, a camera 240 adapted to capture an image of the interior of the auditory canal is attached. To a tip end of the suction tube 230, a dust collecting tube 232 adapted to suck foreign matters in the interior of the auditory canal is attached.

As illustrated in FIGS. 1 to 3, from a tip end on a rear side of the main body unit 200, a cable 225 connected to the camera 240 of the image capturing tube 220 is extracted, and an end portion 231 of the suction tube 230 passing through the main body unit 200 is projected as illustrated in FIG. 7. FIGS. 1 to 3 illustrate a state in which a tube 265 is connected to the end portion 231 of the suction tube 230 (refer to FIG. 7). To a tip end of the cable 225, a USB connector 260 (registered trademark) serving as a connecting tool is attached, and the camera 240 and an image processing section 310 of the control unit 300 are connected via the USB connector 260. Also, the tube 265 is connected to a suction pump 330 of the control unit 300.

As illustrated in FIGS. 1 to 4, the image capturing tube 220 extends from a center of the tip end portion 210 in a longitudinal direction of the main body unit 200. Also, the suction tube 230 extends from a position out of the center of the tip end portion 210 in the longitudinal direction of the main body unit 200 to be adjacent to the image capturing tube 220.

As illustrated in FIG. 4, a projecting length of the suction tube 230 from the tip end portion 210 is shorter than a projecting length of the image capturing tube 220 from the tip end portion 210. To the tip end of the suction tube 230, the dust collecting tube 232 structured as illustrated in FIG. 4 is attached. FIGS. 1 to 3 illustrate a state in which the dust collecting tube 232 is attached to the suction tube 230.

As illustrated in FIGS. 1 to 3, in the state in which the dust collecting tube 232 is attached, a projecting length of the dust collecting tube 232 from the tip end portion 210 is longer than the projecting length of the image capturing tube 220 from the tip end portion 210. This enables the camera 240 of the image capturing tube 220 to capture images of a tip end part of the dust collecting tube 232.

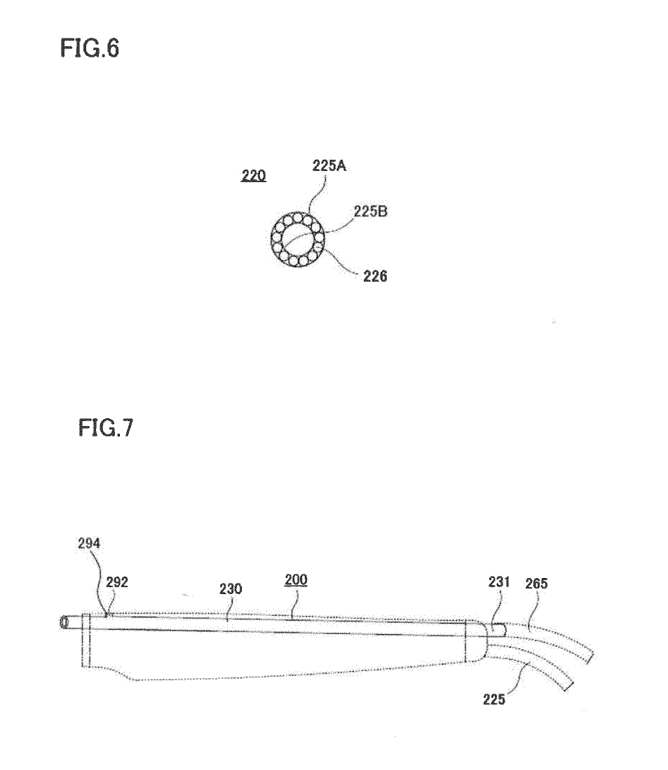

As illustrated in FIG. 6, the image capturing tube 220 has a double-pipe structure in which an outer circumferential pipe 225A has inserted therein an inner circumferential pipe 225B having a shorter outside diameter than an inside diameter of the outer circumferential pipe 225A. Between the outer circumferential pipe 225A and the inner circumferential pipe 225B, a plurality of linear optical fibers 226 serving as a light source illuminating the interior of the auditory canal are arranged along an outer circumference of the inner circumferential pipe 225B. Light guided inside the optical fibers 226 is emitted cylindrically from a tip end part of the image capturing tube 220 to illuminate the interior of the auditory canal.

As illustrated in FIGS. 1 to 4, the tip end side of the image capturing tube 220, more specifically, the tip end side in the inner circumferential pipe 225B, is provided with the camera 240 capturing images of the interior of the auditory canal. Also, a rear side of the camera 240 of the image capturing tube 220, more specifically, a rear side of the camera 240 in the inner circumferential pipe 225B (the side of the tip end portion 210), is provided with a heater 245 or a light source heating body heating the camera 240. A light source supplying the optical fibers 226 (refer to FIG. 6) with light for illuminating the interior of the auditory canal is built in the main body unit 200.

The heater 245 is a resistance heater or a light source heating body adapted to heat the camera 240. By preheating the camera 240 before using the auditory canal cleaning tool 100, misting of a lens of the camera 240 can be prevented when the camera 240 is inserted into the interior of the auditory canal, in which the temperature is higher than outside. Also, by heating the camera 240 continuously, misting of the lens caused by the humidity of the interior of the auditory canal can be prevented when the interior of the auditory canal is observed for a long time.

As illustrated in FIG. 4, the dust collecting tube 232 includes an inserting portion 235 into which the image capturing tube 220 is to be inserted. By having the suction tube 230 inserted into one end of the dust collecting tube 232 and having the image capturing tube 220 inserted into the entire inserting portion 235, the dust collecting tube 232 is attached to the suction tube 230 in a fixed manner.

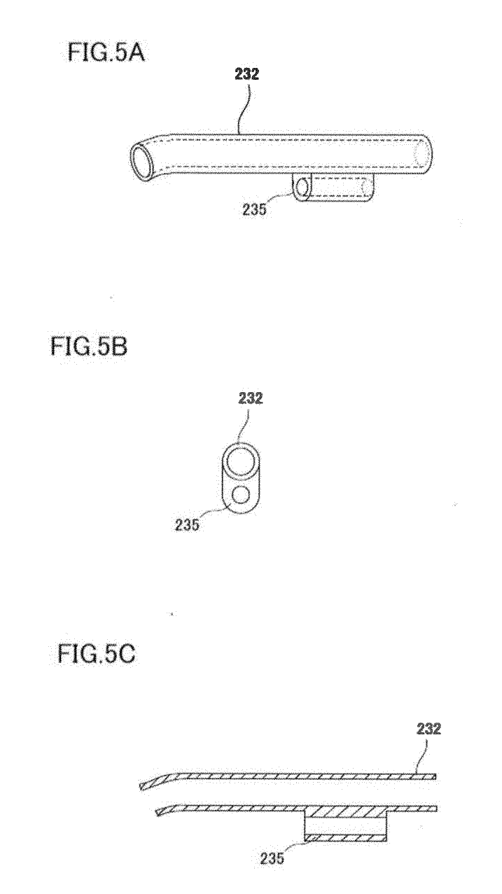

The dust collecting tube 232 is made of flexible and soft vinyl or plastic that does not damage the auditory canal as illustrated in FIG. 5A-5C. The dust collecting tube 232 is replaced each time the auditory canal cleaning tool 100 is used. This means the dust collecting tube 232 is disposable.

The inserting portion 235 is formed integrally with the dust collecting tube 232. The inserting portion 235 functions as an anti-rotating portion preventing the dust collecting tube 232 from being rotated in a circumferential direction of the suction tube 230 and as a reinforcing material reinforcing the strength of the dust collecting tube 232.

An inside diameter of the dust collecting tube 232 is formed to be slightly shorter than an outside diameter of the suction tube 230. An inside diameter of the inserting portion 235 is also formed to be slightly shorter than an outside diameter of the image capturing tube 220.

Thus, by having the suction tube 230 and the image capturing tube 220 inserted into the dust collecting tube 232 and the inserting portion 235, respectively, the dust collecting tube 232 is firmly fixed along the image capturing tube 220.

The dust collecting tube 232 has a short tube diameter enough to enter the interior of the auditory canal and to suck cerumen and is chamfered at the tip end portion thereof not to damage the auditory canal. Since the tube diameter of the dust collecting tube 232 is short, a large foreign matter in the auditory canal is not sucked into the dust collecting tube 232 but attached to the tip end of the dust collecting tube 232 and removed. As a matter of course, a smaller foreign matter in the auditory canal than the tube diameter of the dust collecting tube 232 is sucked into the suction tube 230 through the dust collecting tube 232. Thus, something that can contain sucked foreign matters may be provided around the end portion 231 (refer to FIG. 7) of the suction tube 230.

As illustrated in FIGS. 1 to 5, the tip end part of the dust collecting tube 232 is curved to a side of a center axis of the image capturing tube 220. Curving the tip end part of the dust collecting tube 232 makes it difficult to generate an acute increase of suction pressure even in a case in which the tip end part of the dust collecting tube 232 contacts a delicate part such as an eardrum. Thus, it is possible to prevent the delicate part such as an eardrum from being damaged. Meanwhile, since the dust collecting tube 232 is not rotated in the circumferential direction of the suction tube 230, the direction of the curve of the dust collecting tube 232 will not be changed in the auditory canal.

As illustrated in FIG. 7, the suction tube 230 passes from the tip end side to the inside of the main body unit 200 and projects to the rear end side. The tube 265 connected to the external suction pump 330 illustrated in FIG. 1 is inserted into a part of the suction tube 230 projecting from the rear end side of the main body unit 200. Thus, the suction pump 330 provided in the control unit 300 sucks foreign matters in the interior of the auditory canal via the suction tube 230 and the dust collecting tube 232.

As illustrated in FIGS. 3 and 7, an external surface of the main body unit 200 has a suction pressure adjusting hole 292 adapted to adjust a suction force of the suction tube 230. The suction pressure adjusting hole 292 communicates with the suction tube 230.

As illustrated in FIG. 8, the suction tube 230 passes through the main body unit 200 and has a communication hole 294 opened at a part corresponding to the suction pressure adjusting hole 292. A path from the communication hole 294 to the suction pressure adjusting hole 292 is in an airtight structure. The suction pressure adjusting hole 292 can be closed by a finger of the operator.

As illustrated in FIG. 3, although the suction pressure adjusting hole 292 is provided close to the tip end portion 210 of the main body unit 200, the suction pressure adjusting hole 292 may be provided on the tip end portion 210 as the case may be in consideration of usability for the operator.

In a case in which the operator uses the auditory canal cleaning tool 100, a suction force of the suction tube 230 increases when the suction pressure adjusting hole 292 is closed by his/her finger and decreases when the suction pressure adjusting hole 292 is opened. Accordingly, the operator can adjust the suction force of the suction tube 230 by closing or not closing the suction pressure adjusting hole 292. The size of the opening diameter of the suction pressure adjusting hole 292 may be considered to enable fine adjustment of the suction pressure.

As illustrated in FIG. 1, the control unit 300 includes the image processing section 310 configured to process an image output from the camera 240, a heater control section 320 configured to adjust a temperature of the heater 245, and the suction pump 330 adapted to suck air in the interior of the auditory canal via the suction tube 230 and the dust collecting tube 232. To the image processing section 310, a display 350 is connected.

The image processing section 310 is a dedicated device configured to process an image from the camera 240, or a television, a personal computer, or a mobile terminal (e.g., a smartphone) having installed therein software configured to process an image from the camera 240. The image of the interior of the auditory canal that has been image-processed in the image processing section 310 is output to the display 350. The display 350 is a display device of the television, the personal computer, or the mobile terminal.

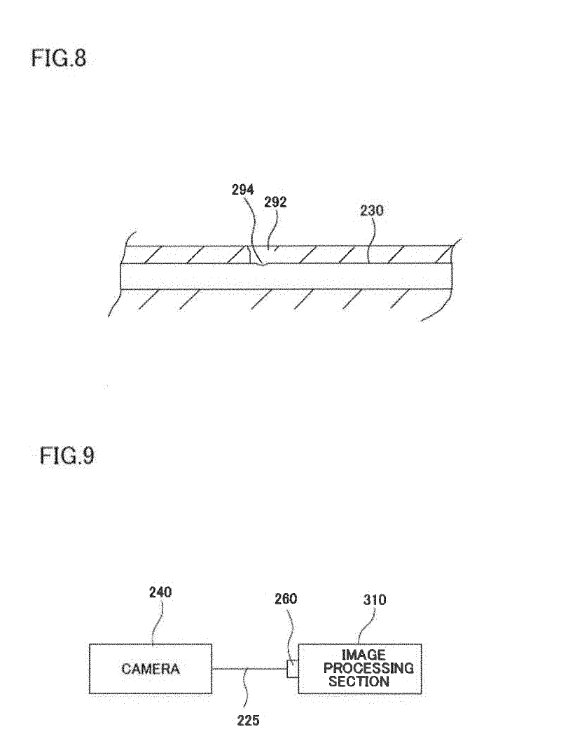

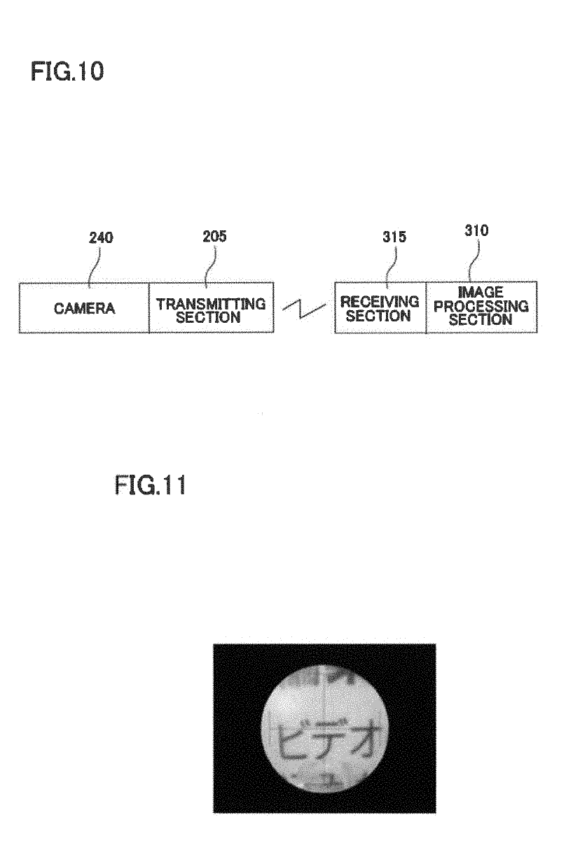

The camera 240 is connected to the external image processing section 310 in a wired or wireless manner. As illustrated in FIG. 9, in a case in which the camera 240 and the image processing section 310 are connected in a wired manner, a cable 225 connected to the camera 240 is connected to the image processing section 310 via the USB connector 260. Also, as illustrated in FIG. 10, in a case in which the camera 240 and the image processing section 310 are connected in a wireless manner, a transmitting section 205 provided in the camera 240 and a receiving section 315 provided in the image processing section 310 are connected via radio waves or light. The image of the interior of the auditory canal that has been image-processed in the image processing section 310 is output to the external display 350, and the operator can look at the image of the interior of the auditory canal as illustrated in FIG. 11, for example.

### [Actions of Auditory Canal Cleaning Tool]

Next, actions of the auditory canal cleaning tool 100 according to Embodiment 1 will be described.

First, the operator attaches the dust collecting tube 232 structured as illustrated in FIG. 5A-5C to the tip end portion 210 of the main body unit 200. The dust collecting tube 232 is attached by having the suction tube 230 inserted into one end of the dust collecting tube 232 and having the image capturing tube 220 inserted into the entire inserting portion 235 as illustrated in FIG. 4.

Subsequently, the operator turns on the power of the control unit 300. The heater control section 320 heats the heater 245 to preheat the camera 240. Also, the suction pump 330 is operated, and air is sucked from the dust collecting tube 232 and the suction pressure adjusting hole 292. Further, an image of the camera 240 is output to the image processing section 310, and an image processed in the image processing section 310 is displayed on the display 350.

The operator then holds the tip end portion 210 of the main body unit 200 as in FIG. 1 and carefully inserts the dust collecting tube 232 attached to the image capturing tube 220 and the suction tube 230 into one's auditory canal.

The operator observes a state of the interior of the auditory canal while looking at the display 350 and turns the dust collecting tube 232 to a foreign matter such as cerumen. The operator closes the suction pressure adjusting hole 292 with his/her finger while moving the dust collecting tube 232 close to the foreign matter to increase suction pressure of the dust collecting tube 232 and attaches the foreign matter to the tip end part of the dust collecting tube 232 or sucks the foreign matter into the dust collecting tube 232.

To take the attached foreign matter out of the auditory canal, the operator separates the main body unit 200 from the ear and pulls the dust collecting tube 232 out of the auditory canal. The operator then releases his/her finger from the suction pressure adjusting hole 292 and removes the foreign matter attached to the dust collecting tube 232. The auditory canal cleaning tool 100 is configured so that the suction pressure of the dust collecting tube 232 may be lower than the suction pressure of the suction pressure adjusting hole 292 when the suction pressure adjusting hole 292 is not closed by his/her finger. Thus, by releasing his/her finger from the suction pressure adjusting hole 292, the attached foreign matter can drop or be removed easily.

The operator repeats the above operation to clean the interior of the auditory canal. When cleaning is finished, the operator detaches the dust collecting tube 232 from the suction tube 230 and the image capturing tube 220 and turns off the power of the control unit 300.

In a case of a small animal, humidity of the interior of its auditory canal is higher than that of a human being. However, since the heater 245 or the light source heating body heats the camera 240, the camera 240 is not misted, and a bright and clear image is displayed on the display 350.

### [Modification Example of Heater of Auditory Canal Cleaning Tool]

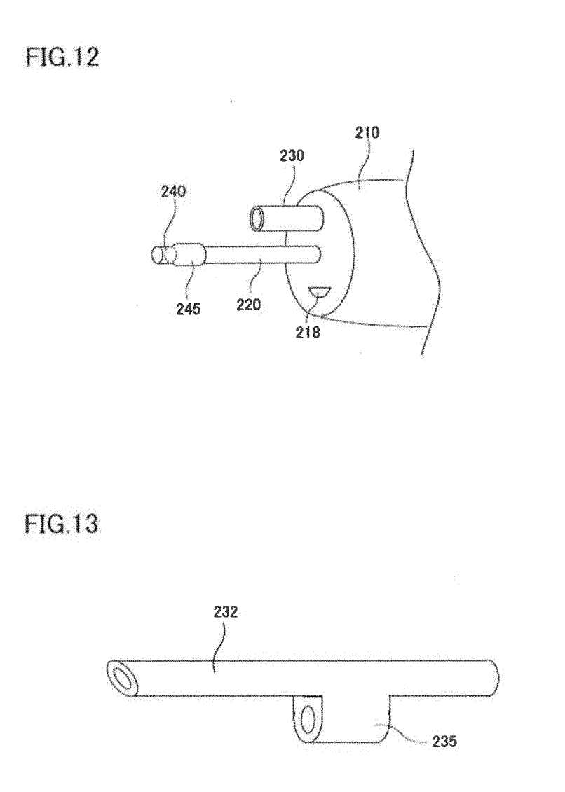

Next, a modification example of the heater 245 described in Embodiment 1 will be described. FIG. 12 illustrates a modification example of the heater 245. The heater 245 illustrated in FIG. 12 is not provided on a rear side of the camera 240 in the inner circumferential pipe 2253 in FIG. 6 as the heater 245 illustrated in FIG. 1 but can be provided to be wound around an outer circumferential portion of the outer circumferential pipe 225A in FIG. 6.

The heater 245 is located on the rear side of the camera 240 in a similar manner to the heater 245 described in Embodiment 1. Although FIG. 12 is an exaggerated view to clearly describe the state in which the heater 245 is wound, the heater 245 actually has an approximately equal outside diameter to that of the image capturing tube since a film-like resistance heater is used.

### [Modification Example of Dust Collecting Tube of Auditory Canal Cleaning Tool]

Next, a modification example of the dust collecting tube 232 described in Embodiment 1 will be described. FIG. 13 illustrates a modification example of the dust collecting tube 232. The dust collecting tube 232 illustrated in FIG. 13 is obliquely cut at the tip end part thereof. Obliquely cutting the tip end part of the dust collecting tube 232 in this manner makes it difficult to generate an acute increase of suction pressure even in a case in which the tip end part of the dust collecting tube 232 contacts a delicate part such as an eardrum. Thus, it is possible to prevent the delicate part such as an eardrum from being damaged.

Meanwhile, although the cut tip end part looks sharply peaked in FIG. 13, the tip end part is actually chamfered as a measure against damage of the interior of the auditory canal.

### Embodiment 2

Embodiment 2 is the auditory canal cleaning tool 100 of a type of not having a function enabling misting of the camera to be prevented as in Embodiment 1. Thus, the auditory canal cleaning tool 100 according to Embodiment 2 only has a function enabling foreign matters in the interior of the auditory canal to be sucked.

### [Configuration of Auditory Canal Cleaning Tool]

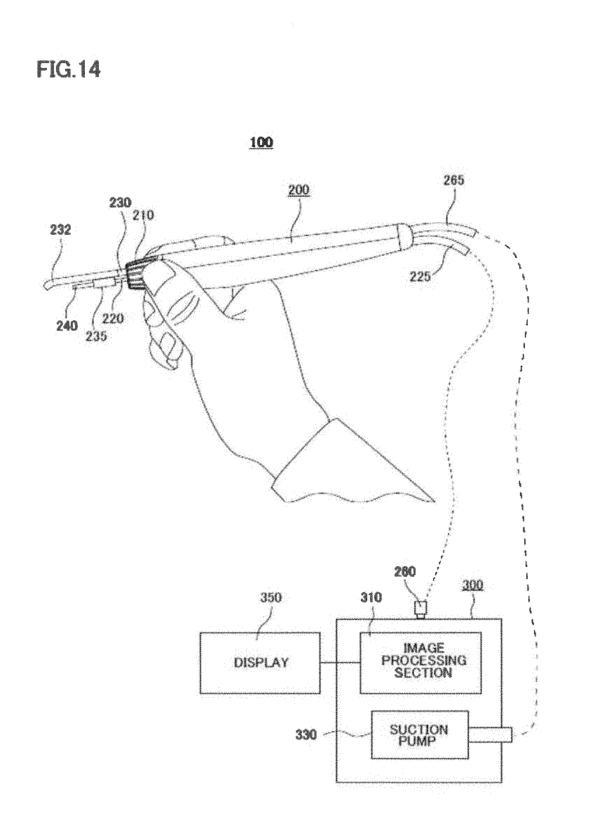

FIG. 14 is a configuration diagram of an auditory canal cleaning tool according to Embodiment 2. As is apparent from comparison between FIG. 1 and FIG. 14, the auditory canal cleaning tool 100 according to Embodiment 2 is the auditory canal cleaning tool 100 according to Embodiment 1 from which the heater 245 and the heater control section 320 are removed.

A configuration of the auditory canal cleaning tool 100 according to Embodiment 2 is equal to that of the auditory canal cleaning tool 100 according to Embodiment 1 except that the heater 245 and the heater control section 320 are removed.

### [Actions of Auditory Canal Cleaning Tool]

Next, actions of the auditory canal cleaning tool 100 according to Embodiment 2 will be described.

First, the operator attaches the dust collecting tube 232 structured as illustrated in FIG. 5A-5C to the tip end portion 210 of the main body unit 200. The dust collecting tube 232 is attached by having the suction tube 230 inserted into one end of the dust collecting tube 232 and having the image capturing tube 220 inserted into the entire inserting portion 235 as illustrated in FIG. 4.

Subsequently, the operator turns on the power of the control unit 300. The suction pump 330 is operated, and air is sucked from the dust collecting tube 232 and the suction pressure adjusting hole 292. Also, an image of the camera 240 is output to the image processing section 310, and an image processed in the image processing section 310 is displayed on the display 350.

The operator then holds the tip end portion 210 of the main body unit 200 as in FIG. 14 and carefully inserts the dust collecting tube 232 attached to the image capturing tube 220 and the suction tube 230 into one's auditory canal.

The operator observes a state of the interior of the auditory canal while looking at the display 350 and turns the dust collecting tube 232 to a foreign matter such as cerumen. The operator closes the suction pressure adjusting hole 292 with his/her finger while moving the dust collecting tube 232 close to the foreign matter to increase suction pressure of the dust collecting tube 232 and attaches the foreign matter to the tip end part of the dust collecting tube 232 or sucks the foreign matter into the dust collecting tube 232.

To take the attached foreign matter out of the auditory canal, the operator separates the main body unit 200 from the ear and pulls the dust collecting tube 232 out of the auditory canal. The operator then releases his/her finger from the suction pressure adjusting hole 292 and removes the foreign matter attached to the dust collecting tube 232. The auditory canal cleaning tool 100 is configured so that the suction pressure of the dust collecting tube 232 may be lower than the suction pressure of the suction pressure adjusting hole 292 when the suction pressure adjusting hole 292 is not closed by his/her finger. Thus, by releasing his/her finger from the suction pressure adjusting hole 292, the attached foreign matter can drop or be removed easily.

The operator repeats the above operation to clean the interior of the auditory canal. When cleaning is finished, the operator detaches the dust collecting tube 232 from the suction tube 230 and the image capturing tube 220 and turns off the power of the control unit 300.

### Embodiment 3

Embodiment 3 is an embodiment of an auditory canal observing tool according to the present invention. The auditory canal observing tool according to Embodiment 3 does not have a function enabling foreign matters in the interior of the auditory canal to be sucked as in Embodiment 1 but only has a function enabling misting of the camera to be prevented.

### [Configuration of Auditory Canal Observing Tool]

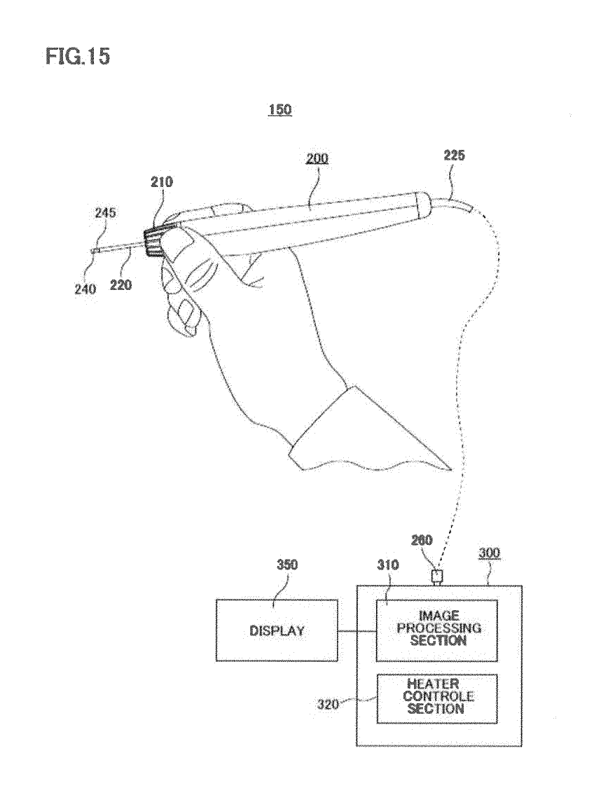

FIG. 15 is a configuration diagram of an auditory canal observing tool. As is apparent from comparison between FIG. 1 and FIG. 15, an auditory canal observing tool 150 is the auditory canal cleaning tool 100 according to Embodiment 1 from which the suction tube 230, the dust collecting tube 232, the tube 265, and the suction pump 330 are removed.

A configuration of the auditory canal observing tool 150 is equal to that of the auditory canal cleaning tool 100 according to Embodiment 1 except that the suction tube 230, the dust collecting tube 232, the tube 265, and the suction pump 330 are removed.

In the auditory canal observing tool 150, the operator can observe the interior of the auditory canal while looking at an image of the interior of the auditory canal clearly displayed on the display 350.

### [Actions of Auditory Canal Observing Tool]

Next, actions of the auditory canal observing tool 150 will be described.

First, the operator turns on the power of the control unit 300. The heater control section 320 heats the heater 245 to preheat the camera 240. Also, an image of the camera 240 is output to the image processing section 310, and an image processed in the image processing section 310 is displayed on the display 350.

The operator then holds the tip end portion 210 of the main body unit 200 as in FIG. 15 and carefully inserts the image capturing tube 220 into one's auditory canal.

The operator observes a state of the interior of the auditory canal while looking at the display 350. When observation is finished, the operator pulls the image capturing tube 220 out of the auditory canal and turns off the power of the control unit 300.

In a case of a small animal, humidity of the interior of its auditory canal is higher than that of a human being. However, since the heater 245 heats the camera 240, the camera 240 is not misted, and a bright and clear image is displayed on the display 350.

### Embodiment 4

Embodiment 4 is an auditory canal observing tool in a state of attaching an ear pick portion and a handle portion thereto. The auditory canal cleaning tool according to Embodiment 4 is the auditory canal cleaning tool 100 according to Embodiment 1 or 2 from which the dust collecting tube 232 is removed and to which the ear pick portion is attached.

### [Configuration of Auditory Canal Cleaning Tool]

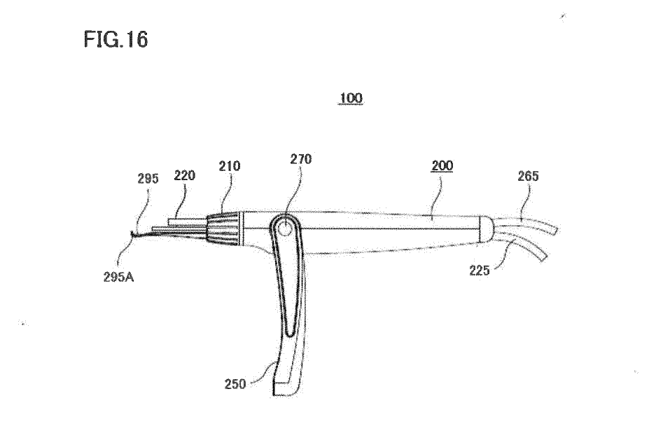

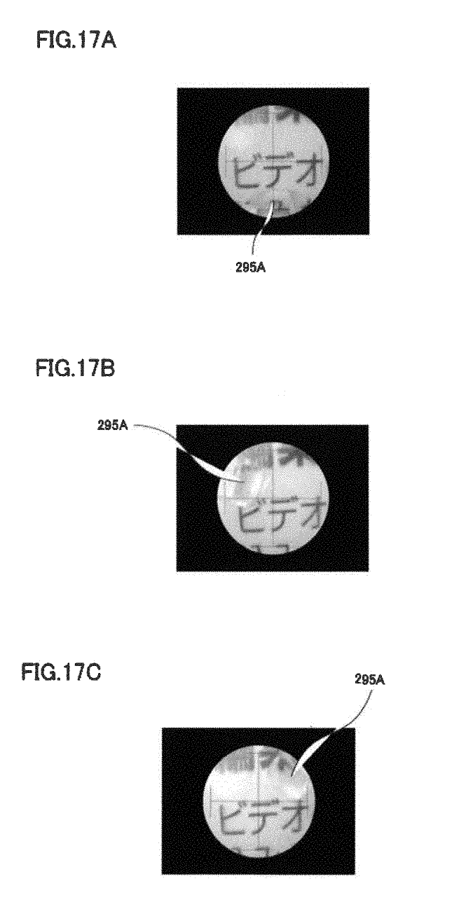

FIG. 16 is a configuration diagram of the auditory canal observing tool in a state of attaching the ear pick portion and the handle portion thereto. FIGS. 17A, 17B and 17C illustrate examples of images obtained by the auditory canal observing tool in FIG. 16.

As is apparent from comparison between FIG. 1 and FIG. 16, the auditory canal cleaning tool 100 is the auditory canal cleaning tool 100 according to Embodiment 1 from which the dust collecting tube 232 is removed and to which an ear pick portion 295 is attached.

To the auditory canal cleaning tool 100, a handle portion 250 supporting the main body unit 200 is attached further on the tip end side than a center of the main body unit 200. The handle portion 250 is attached to be turnable around a fixing tool 270. When the auditory canal cleaning tool 100 is in use, the handle portion 250 is erected from the main body unit 200 as in FIG. 16.

Since the auditory canal cleaning tool 100 according to Embodiment 4 is provided with the handle portion 250, usability is improved when one cleans his/her own ear.

### [Actions of Auditory Canal Cleaning Tool]

Next, actions of the auditory canal cleaning tool 100 will be described.

First, the operator inserts one end of the ear pick portion 295 in FIG. 16 into the ear pick portion attaching hole 218 illustrated in FIG. 4 to firmly attach the ear pick portion 295 to the tip end portion 210.

The ear pick portion attaching hole 218 is formed in a semi-circular shape, and one end of the ear pick portion 295 is also formed in a shape corresponding to that of the ear pick portion attaching hole 218. This prevents axial rotation of the ear pick portion 295, and the direction of a scraping portion 295A is fixed to face a side of the image capturing tube 220.

Subsequently, the operator turns on the power of the control unit 300 illustrated in FIG. 1. The heater control section 320 illustrated in FIG. 1 heats the heater to preheat the camera. The operator then erects the handle portion 250 as illustrated in FIG. 16 and grips the main body unit 200. The handle portion 250 is fixed by the ball of the thumb, the middle finger, the ring finger, and the little finger of the operator and the posture of the main body unit 200 is fixed.

The camera is set to cause an image of a tip end part of the scraping portion 295A located at one end of the ear pick portion 295 to be partially viewed in an image of the interior of the auditory canal. Thus, as illustrated in FIGS. 17A, 17B and 17C, the scraping portion 295A is viewed on the display 350, and the operator can find out in which direction the scraping portion 295A faces.

The operator cleans the interior of his/her auditory canal while looking at an image of the interior of the auditory canal displayed on the display.

When cleaning of the interior of the auditory canal is finished, the operator pulls the ear pick portion 295 out of the auditory canal and turns off the power of the control unit 300.

In a case of a small animal, humidity of the interior of its auditory canal is higher than that of a human being. However, since the heater 245 heats the camera 240, the camera 240 is not misted, and a bright and clear image is displayed on the display 350.

As described above, with the auditory canal cleaning tool according to the present invention, foreign matters of variable size in the interior of one's auditory canal can be removed infallibly. Also, with the auditory canal observing tool according to the present invention, the interior of the auditory canal can be observed on a bright image even in a case in which the interior of the auditory canal is humid.

What is claimed is:

1. An auditory canal cleaning tool comprising:
an image capturing tube adapted to be inserted into one's auditory canal to output an image of an interior of the auditory canal;
a suction tube provided along the image capturing tube to suck a foreign matter in the interior of the auditory canal; and
a main body unit provided with the image capturing tube and the suction tube,
wherein the image capturing tube includes a camera adapted to capture the image of the interior of the auditory canal, and to a tip end of the suction tube, a dust collecting tube is attached.

2. The auditory canal cleaning tool according to claim 1, wherein the dust collecting tube includes an inserting portion into which the image capturing tube is to be inserted, and by having the suction tube inserted into one end of the dust collecting tube and having the image capturing tube inserted into the inserting portion, the dust collecting tube is attached to the suction tube in a fixed manner.

3. The auditory canal cleaning tool according to claim 1, wherein
the suction tube passes from a front side to an inside of the main body unit and projects to a rear side, and a tube connected to an external suction pump is inserted into a projecting part of the suction tube to the rear side,
an external surface of the main body unit has a suction pressure adjusting hole adapted to adjust a suction force of the suction tube, and
the suction pressure adjusting hole communicates with the suction tube.

4. The auditory canal cleaning tool according to claim 1, wherein
the image capturing tube has a double-pipe structure in which an outer circumferential pipe has inserted therein an inner circumferential pipe having a shorter outside diameter than an inside diameter of the outer circumferential pipe,
between the outer circumferential pipe and the inner circumferential pipe, a plurality of linear optical fibers illuminating the interior of the auditory canal are arranged along an outer circumference of the inner circumferential pipe,
the camera is arranged in the inner circumferential pipe, and
the main body unit has built therein a light source supplying the optical fibers with light for illuminating the interior of the auditory canal.

5. The auditory canal cleaning tool according to claim 4, wherein a heater heating the camera is arranged in the inner circumferential pipe or at an outer circumference of the outer circumferential pipe.

6. The auditory canal cleaning tool according to claim 5, wherein the heater is connected to an external heater control section, and a temperature of the heater is controlled by the heater control section.

7. The auditory canal cleaning tool according to claim 1, wherein the camera is connected to an external image processing section in a wired or wireless manner.

8. The auditory canal cleaning tool according to claim 7, wherein, in a case in which the camera and the image processing section are connected in a wired manner, a cable connected to the camera is connected to the image processing section via a connecting tool, and in a case in which the camera and the image processing section are connected in a wireless manner, a transmitting section provided in the camera and a receiving section provided in the image processing section are connected via a radio wave or light.

9. The auditory canal cleaning tool according to claim 1, wherein, to the main body unit, an ear pick portion scraping out the foreign matter in the interior of the auditory canal can be attached, and the camera causes an image of a tip end part of a scraping portion located at one end of the ear pick portion to be partially viewed in the image of the interior of the auditory canal.

10. The auditory canal cleaning tool according to claim 1, wherein the main body unit includes a handle portion supporting the main body unit.

11. An auditory canal observing tool comprising:
an image capturing tube adapted to be inserted into one's auditory canal to output an image of an interior of the auditory canal; and
a main body unit provided with the image capturing tube,
wherein the image capturing tube includes a camera adapted to capture the image of the interior of the auditory canal and a heater heating the camera.

12. The auditory canal observing tool according to claim 11, wherein
the image capturing tube has a double-pipe structure in which an outer circumferential pipe has inserted therein an inner circumferential pipe having a shorter outside diameter than an inside diameter of the outer circumferential pipe,
between the outer circumferential pipe and the inner circumferential pipe, a plurality of linear optical fibers illuminating the interior of the auditory canal are arranged along an outer circumference of the inner circumferential pipe, and
the camera is arranged in the inner circumferential pipe.

13. The auditory canal observing tool according to claim 12, wherein the heater is arranged in the inner circumferential pipe or at an outer circumference of the outer circumferential pipe.

14. The auditory canal observing tool according to claim 11, wherein the heater is connected to an external heater control section, and a temperature of the heater is controlled by the heater control section.

15. The auditory canal observing tool according to claim 13, wherein the main body unit has built therein a light source supplying the optical fibers with light for illuminating the interior of the auditory canal.

16. The auditory canal observing tool according to claim 11, wherein the camera is connected to an external image processing section in a wired or wireless manner.

17. The auditory canal observing tool according to claim 16, wherein, in a case in which the camera and the image processing section are connected in a wired manner, a cable connected to the camera is connected to the image processing section via a connecting tool, and in a case in which the camera and the image processing section are connected in a wireless manner, a transmitting section provided in the camera and a receiving section provided in the image processing section are connected via a radio wave or light.
